(19) United States
(12) Patent Application Publication
(10) Pub. No.: US 2024/0261246 A1
(43) Pub. Date: Aug. 8, 2024

(54) USE OF SMALL MOLECULE COMPOUND AND LENALIDOMIDE IN PREPARATION OF DRUG FOR TREATING MULTIPLE MYELOMA

(71) Applicant: Soochow University, Wujiang (CN)

(72) Inventor: Liang ZHOU, Wujiang (CN)

(21) Appl. No.: 18/638,894

(22) Filed: Apr. 18, 2024

Related U.S. Application Data
(63) Continuation of application No. PCT/CN2023/103672, filed on Jun. 29, 2023.

(30) Foreign Application Priority Data
Oct. 20, 2022 (CN) 202211289198.0

(51) Int. Cl. A61K 31/20; A61K 31/454; A61P 35/00

(52) U.S. Cl. CPC A61K 31/20; A61K 31/454; A61P 35/00

(57) ABSTRACT

The invention relates to the field of drugs, and particularly to use of a small molecule compound and lenalidomide in the preparation of a drug for treating multiple myeloma. How to promote the degradation of a transcription factor IKZF1 necessary for the proliferation of a downstream substrate, namely myeloma cells, is a difficulty in the treatment of multiple myeloma. According to the present invention, the combined use of the small molecule compound 2-BP and lenalidomide in the treatment of multiple myeloma obviously reduces the IKZF1 protein level, and the proliferation of the multiple myeloma cells is inhibited after the multiple myeloma cells are treated by the small molecule compound 2-BP in combination with lenalidomide.

### FIELD OF THE INVENTION

The present invention relates to the field of drugs, and particularly to use of a small molecule compound and lenalidomide in the preparation of a drug for treating multiple myeloma.

### DESCRIPTION OF THE RELATED ART

Multiple myeloma (MM) is a blood cancer caused by the malignancy of plasma cells. MM is refractory due to the drug resistance and recurrence, and other characteristics.

In the disease, the tumor cells are originated from plasma cells in bone marrow, which are cells that B lymphocytes develop to the final functional stage. Therefore, multiple myeloma can be deemed as B-lymphocyte lymphoma. At present, it is classified as a B-cell lymphoma, called plasma cell myeloma/plasma cell neoplasm. MM is characterized by abnormal proliferation of bone marrow plasma cells accompanied by the overproduction of monoclonal immunoglobulin or light chain (M protein), and very few patients may have non-secretory multiple myeloma that does not produce M protein. Multiple myeloma is often accompanied by multiple osteolytic lesions, hypercalcemia, anemia and kidney damage. Because the production of normal immunoglobulin is inhibited, various bacterial infections tend to occur.

Multiple myeloma is developed slowly and has no obvious symptoms in the early stage. The clinical manifestations of multiple myeloma are diverse, including anemia, bone pain, renal insufficiency, infections, bleeding, neurological symptoms, hypercalcemia, and amyloidosis. In case of bone pain, bone deformation and pathological fracture, myeloma cells secrete osteoclast activating factor which activates osteoclasts, causing the bone dissolution and destruction. Bone pain is the most common symptom, and mostly lumbosacral, sternum and rib pain. Due to the destruction of the bone by tumor cells, pathological fractures may occur, and multiple fractures can exist at the same time. In case of anemia and bleeding, anemia is common, and is an initial symptom. Anemia is mild in the early stage and serious in the later stage. Thrombocytopenia can occur in the advanced stage, causing bleeding symptoms. Skin and mucosal bleeding is common, and visceral and intracranial bleeding may occur in severe cases. Liver, spleen, lymph nodes and kidney pathologies, liver and spleen enlargement, cervical lymph node enlargement, and myeloma kidney are developed in some cases. Extramedullary plasmacytoma or amyloidosis should be considered in case of organ enlargement or abnormal mass.

Extramedullary plasmacytoma of nervous system can cause limb paralysis, lethargy, coma, diplopia, blindness and vision loss. Multiple myeloma is often accompanied by bacterial infection, and also fungal and viral infection. Bacterial pneumonia, urinary tract infection and sepsis are most common. Viral herpes zoster also tends to occur, especially in patients with low immunity after treatment. For renal impairment, proteins, red blood cells, white blood cells and casts are detected in the urine of 50%-70% of patients. Chronic renal failure, hyperphosphatemia, hypercalcemia, and hyperuricemia are developed, and uric acid stones are formed. MM is refractory due to the drug resistance and recurrence, and other characteristics.

The development of lenalidomide (Len) and other immunomodulatory drugs has greatly prolonged the survival of patients with multiple myeloma.

Lenalidomide is an analog of thalidomide, having immunomodulatory, antiangiogenic and antitumor effects. The activity of lenalidomide in cells is mediated by its target protein cereblon. In in-vitro experiments, in the presence of the drug, the substrate protein is degraded after targeted ubiquitination, which leads to direct cytotoxicity and immunomodulation. Lenalidomide can inhibit the proliferation and promote the apoptosis of some hematopoietic tumor cells (including multiple myeloma and mantle cell lymphoma) in vitro. Lenalidomide can delay the tumor growth of some non-clinical hematological tumor models (including multiple myeloma) in vivo. The immunomodulatory effects of lenalidomide include increasing the number and activity of T cells and natural killer cells, and directly enhancing antibody-dependent cell-mediated cytotoxicity (ADCC) by increasing the secretion of IL-2 and INF-7, increasing the number of natural killer T cells and inhibiting the release of proinflammatory cytokines from monocytes. The combination of lenalidomide and dexamethasone can synergistically inhibit the proliferation and induce the apoptosis of multiple myeloma cells. In in-vitro experiments, compared with rituximab alone, the combination of lenalidomide and rituximab can enhance the ADCC effect on follicular lymphoma cells and cause the direct apoptosis of tumor cells, and can enhance the ADCC effect on marginal lymphoma cells.

However, the resistance to a single drug still limits the use of this kind of drugs. Lenalidomide degrades the downstream substrate IKZF1 through its binding protein cereblon (CRBN). IKZF1 is an essential transcription factor for the proliferation of myeloma cells. Therefore, Lenalidomide inhibits the proliferation of multiple myeloma cells through CRBN-IKZF1, thus achieving the purpose of treating multiple myeloma.

Lenalidomide has a good effect in the treatment of multiple myeloma. However, the resistance to a single drug still limits the use of this kind of drugs. In recent years, the combination therapy based on lenalidomide has become the first choice to treat multiple myeloma. However, such diseases are prone to relapse and development of drug resistance, making it difficult and hot to develop new mechanisms and strategies for combination therapy against such diseases.

After long-term treatment with lenalidomide, the level of CRBN protein in patients with multiple myeloma decreases, and resistance to lenalidomide occurs in the patients. How to promote the degradation of a transcription factor IKZF1 necessary for the proliferation of a downstream substrate, namely myeloma cells, is a difficulty in the treatment of multiple myeloma.

### SUMMARY OF THE INVENTION

To solve the problems existing in the prior art, the present invention provides use of a small molecule compound and lenalidomide in the preparation of a drug for treating multiple myeloma. The small molecule compound is 2-BP (2-bromopalmitate, CAS: 18263-25-7).

Preferably, the drug is used to reduce the IKZF1 protein level.

Preferably, the drug is used to improve the sensitivity of multiple myeloma cells to lenalidomide.

The present invention further provides a drug for preventing and/or treating multiple myeloma, which includes the small molecule compound 2-BP and lenalidomide.

Preferably, the drug further includes a pharmaceutically acceptable carrier.

Particularly, the carrier is selected from the group consisting of a tablet, a capsule, a pill, a powder, a suppository, an ointment, a solution, and a suspension.

Preferably, the drug further includes an additive.

Particularly, the additive is selected from the group consisting of an antioxidant, a preservative, a solubilizer, a disintegrant, a lubricant, a colorant, a dispersant, a surfactant and any combination thereof.

The most common additive in tablets is corn starch. The corn starch added in oral tablets is a filler, also called diluent, which mainly functions to increase the weight or volume of a drug, so as to facilitate the compression of the drug into tablets.

Tablets are generally required to have a diameter that is not less than 6 mm, and a weight of 100 mg or more. If the main drug in the tablet is only a few milligrams or dozens of milligrams, it will not be prepared into tablets. The volume can be increased by adding an appropriate filler, so that the weight is enough to facilitate the formation of the tablets. In addition to corn starch, the commonly used fillers also include sugars, dextrin, mannitol or the like. A tablet often contains a variety of fillers. For example, corn starch is often mixed with powdered sugar and dextrin with good compressibility. This is because the compressibility of starch is poor, and if it is used alone, the compressed tablets will be too loose.

Fillers, such as mannitol, absorb heat and provide a cool feeling when dissolved in the mouth, which can be used to prepare chewable tablets and can be used in conjunction with sucrose.

Preferably, the drug is administered at a dose of 1-100 mg/kg.

Preferably, the drug is administered orally, intraperitoneally, subcutaneously, intravenously or intramuscularly.

Oral administration is the most important mode of administration. Drugs can be absorbed by the digestive tract after oral administration. The surface area of intestinal mucosa is very large, and drugs are generally absorbed in the small intestine. Only a small number of drugs can be absorbed in the stomach, which are usually some acidic drugs.

Where the drugs cannot be administered orally or through the digestive tract, parenteral administration will be chosen.

Oral administration is inappropriate for many patients with coma and vomiting. Some drugs are poorly absorbed in the digestive tract and are easily destroyed due to various factors. In this case, injection is considered for administration. There are two main ways, one is intramuscular injection, that is, injection into the muscle, and the other is infusion, that is, intravenous injection. Generally speaking, the prerequisite for intramuscular injection is that the drug is highly water-soluble, and can be dissolved in the fluid of muscle stroma, so that the drug can be absorbed quickly. Generally, the injection site is gluteus maximus or deltoid muscle in the shoulder.

It should be noted that intramuscular injection has disadvantages for shock patients. The blood flow under the skin and in the muscles of these patients is very small, so the absorption after local intramuscular injection of drugs is very low. In this case, intravenous administration is considered.

Compared with the prior art, the technical solution of the present invention has the following advantages.

How to promote the degradation of a transcription factor IKZF1 necessary for the proliferation of a downstream substrate, namely myeloma cells, is a difficulty in the treatment of multiple myeloma. According to the present invention, the combined use of the small molecule compound 2-BP and lenalidomide in the treatment of multiple myeloma obviously reduces the IKZF1 protein level, and the proliferation of the multiple myeloma cells is inhibited after the multiple myeloma cells are treated by the combination with lenalidomide.

### BRIEF DESCRIPTION OF THE DRAWINGS

FIG. 1 shows the change of IKZF1 protein level detected by western blot.

FIG. 2 shows the inhibitory effect of lenalidomide on the proliferation of multiple myeloma cells.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

### Example 1

In Example 1, multiple myeloma cells were treated with the small molecule compound 2-BP (purchased from Selleck) in combination with lenalidomide (Len, purchased from MedChemExpress), to reduce the level of IKZF1 protein.

The commercially available RPMI8226 and LP1 cells were inoculated on a 24-well plate. The RPMI8226 and LP1 cells were pretreated with 2-BP (10 μM) for 1 h, and then treated with lenalidomide (10 μM) for 12 h. Then the cells were harvested and lysed, and the change of IKZF1 protein level was detected by western blot.

The specific process for western blot protein was as follows. The cells are lysed with RIPA lysing buffer (150 mM Nacl, 1% Triton x-100, 0.1% SDS, 50 mM Tris-base (pH 7.4), 1 mM EDTA, Protein inhibitors (Bimake)), and then ultrasonically crushed. 5×SDS-PAGE loading buffer (250 mM Tris-HCL (pH 6.8), 10% SDS, 0.25% bromophenol blue (BPB), 50% glycerol, and 5% β-mercaptoethanol) was added for treatment at 98° C. for 5-10 min. SDS-PAGE electrophoresis was carried out with 10% protein gel to isolate the protein. After transmembrane, the protein was blocked with 10% skimmed milk (3 g skimmed milk powder, 30 ml 1×TBST buffer) for 1 h, and then incubated with corresponding primary antibody and secondary antibody. Finally, pictures were taken by the gel imaging system Tanon 5200.

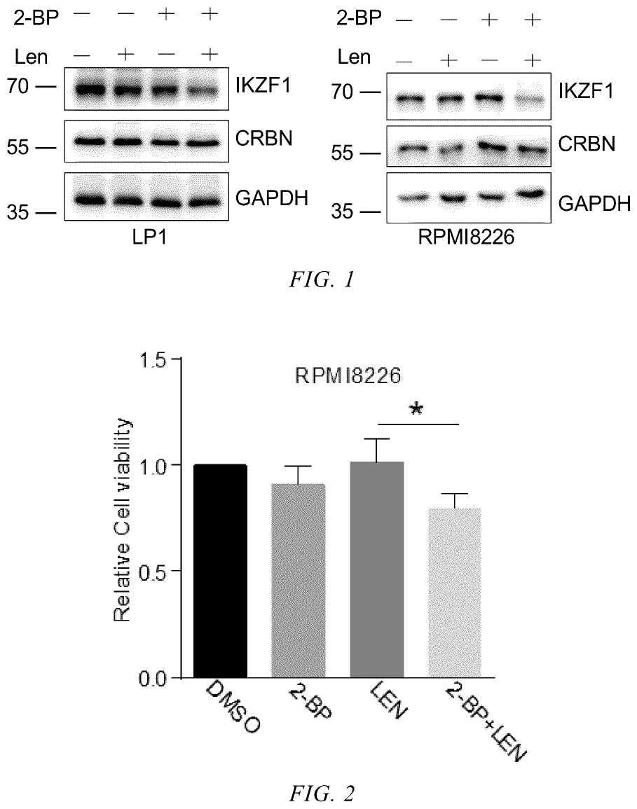

It can be seen from FIG. 1 that 2-BP combined with lenalidomide can significantly down-regulate the protein level of IKZF1.

### Example 2

Example 2 is provided for verifying that the small molecule compound 2-BP can significantly improve the inhibitory effect of lenalidomide on the proliferation of multiple myeloma cells.

RPMI8226 myeloma cells were inoculated on a 24-well plate, pre-treated with 2-BP (10 μM) for 1 h, and then treated with LEN (10 μM) for 5 days. The cell proliferation was detected by using the cell proliferation activity kit CCK8 (purchased from Beyotime Biotechnology).

Cell Counting Kit 8, abbreviated as CCK-8 kit, is a highly sensitive and non-radioactive colorimetric approach for determining the number of living cells in cell proliferation or toxicity experiments.

CCK-8 is an upgraded product of MTT. The CCK-8 solution can be directly added to cell samples without pre-preparation of various components, which can achieve quick detection with low toxicity. CCK-8 is based on water-soluble tetrazolium salt WST-8. The working principle is as follows: WST-8 can be reduced by the dehydrogenase in mitochondria in the presence of an electron coupling reagent to produce an orange-yellow formazan dye. The formazan dye can be dissolved in a tissue culture medium, and is proportional to the living cell counts. By colorimetry, the living cell counts can be dynamically quantified, so as to detect the cell proliferation or drug toxicity. This kit is usually used to detect the cycle and apoptosis of adherent or suspended cells. If used in a tissue, the tissue needs to be digested into single cells before detection.

As can be seen in FIG. 2, the use of the small molecule compound 2-BP and LEN in combination can significantly inhibit the proliferation of myeloma cells.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. Use of a small molecule compound and lenalidomide in the preparation of a drug for treating multiple myeloma, wherein the small molecule compound is 2-BP.

2. The use according to claim 1, wherein the drug is used to reduce a IKZF1 protein level.

3. The use according to claim 1, wherein the drug is used to improve the sensitivity of multiple myeloma cells to lenalidomide.

4. A drug for preventing and/or treating multiple myeloma, comprising the small molecule compound of claim 1 and lenalidomide.

5. The drug according to claim 4, further comprising a pharmaceutically acceptable carrier.

6. The drug according to claim 5, wherein the carrier is selected from the group consisting of a tablet, a capsule, a pill, a powder, a suppository, an ointment, a solution, and a suspension.

7. The drug according to claim 4, further comprising an additive.

8. The drug according to claim 7, wherein the additive is selected from the group consisting of an antioxidant, a preservative, a solubilizer, a disintegrant, a lubricant, a colorant, a dispersant, a surfactant and any combination thereof.

9. The drug according to claim 4, wherein the drug is administered at a dose of 1-100 mg/kg.

10. The drug according to claim 4, wherein the drug is administered orally, intraperitoneally, subcutaneously, intravenously, or intramuscularly.
